(19) United States
(12) Patent Application Publication
(10) Pub. No.: US 2020/0367855 A1
(43) Pub. Date: Nov. 26, 2020

(54) BREAST SLIP PREVENTION PAD FOR ULTRASOUND BREAST SCANNER

(71) Applicant: MEDICALPARK CO.,LTD., Yongin-si, Gyeonggi-do (KR)

(72) Inventor: Hee Boong PARK, Seoul (KR)

(21) Appl. No.: 16/463,983

(22) PCT Filed: Nov. 23, 2017
(86) PCT No.: PCT/KR2017/013394

§ 371 Date: May 24, 2019

(30) Foreign Application Priority Data
Nov. 25, 2016 (KR) 10-2016-0158718

(51) Int. Cl. A61B 8/00

(52) U.S. Cl. CPC A61B 8/406; A61B 8/461; A61B 8/403

(57) ABSTRACT

Provided is a breast slip prevention pad for preventing a breast of a patient from slipping between compression presses when using a compression press type ultrasonic breast scanner. The breast slip prevention pad for an ultrasonic breast scanner includes a mesh sheet formed of an ultrasonic-wave-permeable flexible synthetic resin, and a support plate fixed to one surface of the mesh sheet to support the mesh sheet in a tightly stretched state so that a part of the mesh sheet is exposed. The mesh sheet includes a plurality of transverse ribs and a plurality of longitudinal ribs arranged to extend in a direction perpendicular to the transverse ribs, and the height of the longitudinal ribs is set to be larger than the height of the transverse ribs.

### TECHNICAL FIELD

The present invention relates to a slip prevention pad for preventing a breast of a patient from slipping between compression presses at the time of using a compression press type ultrasonic breast scanner.

### BACKGROUND ART

An ultrasonic scanning device is used to diagnose a woman breast cancer. When scanning is performed to obtain ultrasonic images of a breast, the breast is placed on the upper surface of a scanning unit. At this time, if an air is present between an ultrasonic probe and a skin surface of the breast making contact with the ultrasonic probe, ultrasonic waves are not transmitted properly into the breast due to attenuation or other causes. That is, the ultrasonic waves are reflected at the interface between the air and the skin. Even if there are very small bubbles, the transmission of the ultrasonic waves is remarkably reduced. This makes it difficult to obtain clear images. Thus, an acoustic coupling medium is interposed between the ultrasonic probe and the breast skin surface so that the ultrasonic waves are transmitted to the breast through the acoustic coupling medium. Accordingly, an ultrasonic wave transmission medium is disposed between the upper surface of the scanning unit and the breast.

In order to improve the accuracy of breast cancer diagnosis in clinical practice, it is necessary for a doctor to compare the breast image obtained by x-ray and the breast image obtained by an ultrasonic examination device. The inventor of the present invention has invented and registered a patent for an ultrasonic examination device capable of obtaining a breast image similar to an X-ray image (see Patent Document 1).

The ultrasonic breast scanner disclosed in Patent Document 1 is of a compression press type, and includes an ultrasonic scanning unit for placing the breast and a compression press for compressing the breast placed on the scanning unit. In particular, the ultrasonic scanning unit includes at least one ultrasonic probe and a caterpillar for moving the ultrasonic probe while supporting an object without causing deformation thereto.

When an image of the breast is obtained using the compression press type device as disclosed in Patent Document 1, the breast is pressed by a compression press and the breast deformed by the compression press is scanned. During the scanning, an ultrasonic wave medium (gel or gel pad) is interposed between the breast and the scanning unit. Thus, the breast placed between the scanning unit and the compression press may slip in some case, failing to obtain a desired breast image.

When a liquid gel is used as the ultrasonic wave medium, there is a problem that the application of the liquid gel to the breast skin surface may be uncomfortable to a patient and the liquid gel may be evaporated or moved to an undesired location, which makes it inconvenient to continuously use the liquid gel. In addition, when the ultrasonic scanner is used by several persons, the gel remaining on the surface of the scanning unit may be contaminated, and the patient may undergo infection due to the contact with the contaminated gel. In order to solve the above-described problem posed in the case of using the liquid gel, an ultrasonic gel pad obtained by crosslinking polymers, which is not a liquid type pad, has been devised (see Patent Document 2). Use of the gel pad solidified by polymer crosslinking rather than a liquid type pad can solve the problem of infection to a certain extent. However, there is a problem that the gel pad may be damaged when repeatedly used to capture images of various parts of the breast. The problem that the breast slips when pressed with the compression press still remains.

Meanwhile, Patent Document 3 discloses a pressure scanning assembly for ultrasonic wave scanning. The pressure scanning assembly includes a sheet for pressing the breast, and the sheet is fixed to a frame. The sheet is tightened when pressed against the patient's breast. The sheet is coated with an ultrasonic wave medium. The ultrasonic probe is configured to scan the breast through the sheet. That is, the sheet shown in Patent Document 3 is not designed to prevent the breast from slipping due to the pressure of the compression press when scanning the breast with ultrasonic waves.

### Patent Documents

Patent Document 1: Korean Patent No. 10-0668766, entitled “Ultrasonic inspection device for deformable object”
Patent Document 2: Korean Patent No. 10-0511341, entitled “Gel pad for ultrasonic wave containing carrageenan”
Patent Document 3: U.S. Pat. No. 8,721,552, entitled “Compressive surfaces for ultrasonic scanning”

### SUMMARY

An object of the present invention is to provide a breast slip prevention pad for preventing a pressed breast from slipping when a compression press type ultrasonic scanner is used.

Another object of the present invention is to provide a breast slip prevention pad containing an ultrasonic wave medium for preventing a patient from being infected by a contaminated ultrasonic wave medium when a scanner is used by a plurality of persons.

The breast slip prevention pad for an ultrasonic breast scanner according to the present invention includes: a mesh sheet formed of an ultrasonic-wave-permeable flexible synthetic resin; and a support plate fixed to one surface of the mesh sheet to support the mesh sheet in a tightly stretched state so that a part of the mesh sheet is exposed. The mesh sheet includes a plurality of transverse ribs and a plurality of longitudinal ribs arranged to extend in a direction perpendicular to the transverse ribs. The height of the longitudinal ribs is set to be larger than the height of the transverse ribs.

Preferably, the transverse ribs of the mesh sheet may be arranged to extend toward the support plate.

When in use, the breast slip prevention pad is mounted on an ultrasonic scanning unit, and the gel is applied to the mesh sheet and the surface of the breast. Ultrasonic wave scanning is performed in a state in which the breast is positioned on the mesh sheet of the breast slip prevention pad and the upper portion of the breast is pressed by a compression press. That is to say, the support plate is positioned opposite to the breast of the patient in a state in which the breast slip prevention pad is mounted on the ultrasonic scanning unit. The surface of the breast placed on the mesh sheet initially makes contact with the longitudinal ribs having a larger height. The lower surface of the breast deformed by the compression press is inserted into between the longitudinal ribs to make contact with some portions of the transverse ribs. Therefore, even when the breast is pressed by the compression press, the breast is caught by the transverse ribs, so that the movement of the breast in the transverse direction is impeded and is prevented from slipping.

In some embodiments, the support plate may be provided with a plurality of through-holes so that when attaching the breast slip prevention pad to the ultrasonic breast scanner, the breast slip prevention pad can be easily mounted at a predetermined position and can be fixed in place.

In some embodiments, if the breast slip prevention pad is packaged by applying a gel to the mesh sheet, the user does not have to apply a gel after mounting the breast slip prevention pad on the ultrasonic wave scanner.

In some embodiments, the breast slip prevention pad for an ultrasonic breast scanner may further include: a gel pad disposed adjacent to the support plate of the mesh sheet. When the breast is mounted on the mesh sheet, the gel pad fills the space between the end portion of the breast and the scanning unit so that the ultrasonic waves can be transmitted to the distal end portion of the breast of the patient. This makes it possible to obtain a clearer image of the breast. Particularly, if the gel pad is configured so as to become thinner as it extends away from the support plate along the transverse ribs, it is possible to make sure that the ultrasonic waves are smoothly transferred to the distal end portion of the breast.

According to the present invention, when using a compression press type ultrasonic breast scanner, it is possible to prevent the breast from moving due to compression, thereby obtaining a clear ultrasonic image. Furthermore, it is possible to prevent the breast from slipping due to compression, thereby obtaining an ultrasonic breast image for a wider area. In addition, use of the disposable breast slip prevention pad according to the present invention makes it possible to prevent infection caused by gel contamination even if an ultrasonic wave scanner is used by a plurality of persons.

### BRIEF DESCRIPTION OF THE DRAWINGS

FIG. 1 is a schematic view showing a state in which a breast slip prevention pad is attached to and used in a compression press type ultrasonic breast scanner.

FIG. 2 is a perspective view for explaining a state in which the breast slip prevention pad is mounted on the scanning unit of the ultrasonic breast scanner.

FIG. 3 is a perspective view for explaining a structure of a mesh of the breast slip prevention pad according to the present invention.

FIG. 4 is a perspective view showing another example of the breast slip prevention pad according to the present invention.

FIG. 5 is an explanatory view showing a use state of the breast slip prevention pad shown in FIG. 4.

FIG. 6 illustrates explanatory views showing various examples in which the breast slip prevention pad according to the present invention is packaged in a disposable manner.

### DETAILED DESCRIPTION

Embodiments of the present invention will now be described with reference to the accompanying drawings.

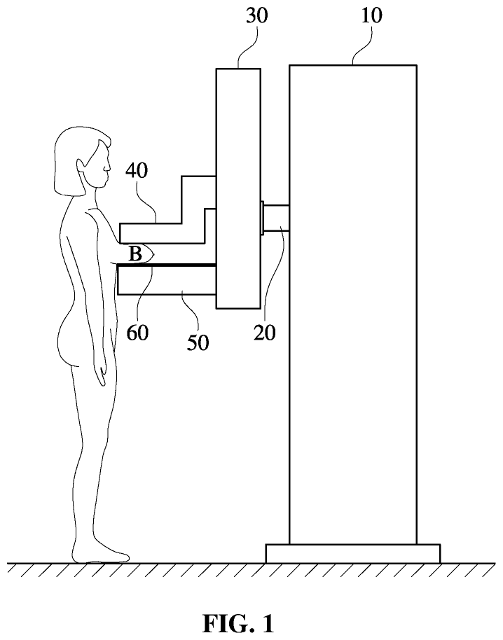

Referring to FIG. 1, the ultrasonic breast scanner 100 includes a scanning unit 50 for supporting a breast of a patient, and a compression press 40 provided at an upper portion of the scanning unit 50 so as to be vertically movable. The scanning unit 50 is fixed to a rotary frame 30, and the compression press 40 is installed so as to be movable up and down with respect to the rotary frame 30. The rotary frame 30 is fixed to a rotary shaft 20, and the rotary shaft 20 is rotatably installed in a main frame 10. A breast slip prevention pad 60 according to the present invention is mounted on the upper surface of the scanning unit 50. There is shown a state in which a breast B of a patient is placed on the breast slip prevention pad 60 and is pressed and deformed by the compression press 40.

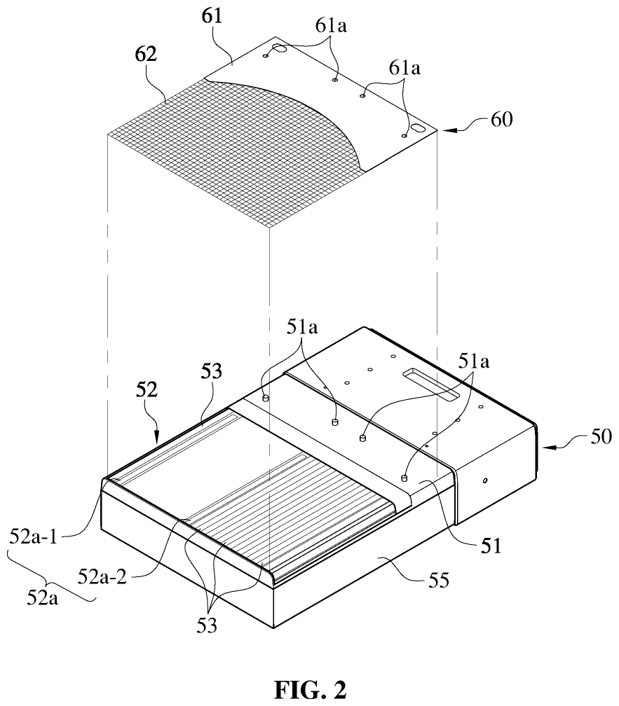

FIG. 2 shows the scanning unit 50 of the ultrasonic breast scanner 100 and the breast slip prevention pad 60 mounted on the scanning unit 50. The scanning unit 50 includes a housing 55 and a caterpillar scanner 52 mounted on the housing 55 so as to constitute a part of an upper surface of the housing 55. The caterpillar scanner 52 includes a plurality of links 53 configured to form a plane when the frame is exposed upward and an ultrasonic probe unit 52a installed between the links 53. The ultrasonic probe unit 52a includes a pair of probes 52a-1 arranged to be spaced apart at a regular interval and a support plate 52a-2 for supporting the pair of probes 52a-1. Although not shown, the links 53 of the caterpillar scanner 52 are configured to move along tracks and to go into or come out from the housing 55 while rotating at the corners of the housing 55. Furthermore, a drive means for driving the probe unit 52a of the caterpillar scanner 52 to move horizontally is installed in the housing 55. Therefore, when the probe unit 52a horizontally moves back and forth, the links 53 goes into or comes out from the housing 55 to scan the breast B placed on the caterpillar scanner 52, thereby obtaining an ultrasonic image. Although not shown, a transparent synthetic resin film is mounted on the upper portion of the caterpillar scanner 52. The transparent synthetic resin film prevents the ultrasonic wave medium from adhering to the links 53 or the probe unit 52a. Furthermore, the inside of the housing 55 is filled with oil or water for ultrasonic wave transmission between the pair of probes 52a-1 and the transparent synthetic resin film. A pad mounting portion 51 for fixing the breast slip prevention pad 60 and determining the mounting position of the breast slip prevention pad 60 is provided above the housing 55 of the scanning unit 50. The pad mounting portion 51 is provided with a plurality of guide projections 51a.

The breast slip prevention pad 60 includes a mesh sheet 62 and a support plate 61. The mesh sheet 62 is made of a flexible synthetic resin which is ultrasonic wave permeable. The support plate 61 is made of a synthetic resin and is fixed to one surface of the mesh sheet 61 so as to support the mesh sheet 62 in a tightly stretched state. Furthermore, the support plate 61 is fixed so that a part of the mesh sheet 62 is exposed on one side of the mesh sheet 62. The support plate 61 is provided with a plurality of through-holes 61a formed at positions corresponding to the projections 51a formed in the pad mounting portion 51 of the upper portion of the housing 55 of the scanning unit 50. The projections of the pad mounting portion 51 are fitted into the corresponding through-holes 61a so that the breast slip prevention pad 60 is mounted at a predetermined position, thereby making sure that when the breast B is pressed by the compression press 40, the breast slip prevention pad 60 does not slip off from the scanning unit 50.

In the present embodiment, the projections are formed in the pad mounting portion 51, and the through-holes 61a are formed in the support plate 61. Conversely, through-holes may be formed in the pad mounting portion 51, and the projections may be formed in the support plate 61. If necessary, a mechanism for fixing the support plate 61 to a predetermined position of the pad mounting portion 51 may be separately provided in the pad mounting portion 51.

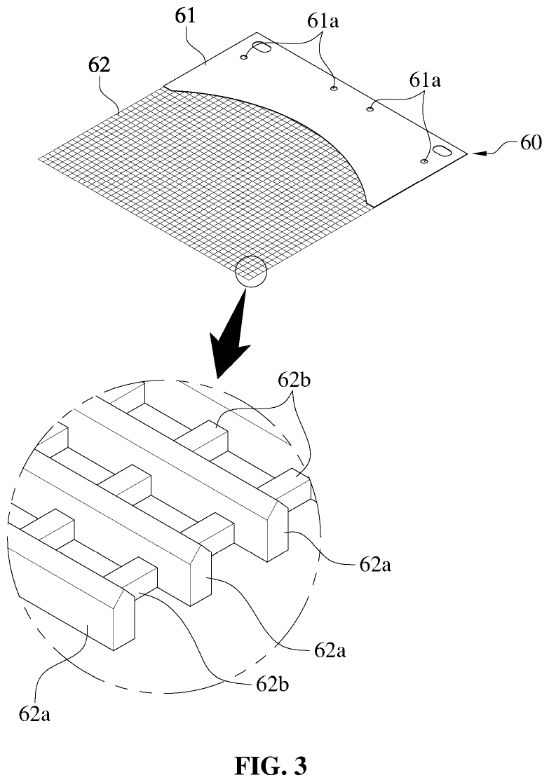

Referring to FIG. 3, the mesh sheet 62 includes a plurality of transverse ribs 62b arranged to extend toward the support plate 61, and a plurality of longitudinal ribs 62a arranged to extend in a direction perpendicular to the transverse ribs 62b. The height of the longitudinal ribs 62a is set to be larger than the height of the transverse ribs 62b so as to prevent the breast B placed on the mesh sheet 62 from slipping.

When in use, the breast slip prevention pad 60 is mounted on the ultrasonic scanning unit 50, and the gel is applied to the mesh sheet 62 and the surface of the breast B. The caterpillar scanner 52 is driven to obtain an image of the breast B in a state in which the breast B is positioned on the mesh sheet 62 of the breast slip prevention pad 60 and the upper portion of the breast B is pressed by the compression press 40. The support plate 61 is positioned opposite to the breast B of the patient in a state in which the breast slip prevention pad 60 is mounted on the ultrasonic scanning unit 50. When the breast B placed on the mesh sheet 60 is pressed by the compression press 40, the surface of the breast B initially makes contact with the longitudinal ribs 62a having a larger height. The lower surface of the breast B deformed by the compression of the compression press 40 is inserted into between the longitudinal ribs 62a to make contact with some portions of the transverse ribs 62b. Therefore, when the breast B is pressed by the compression press 40, the breast B is caught by the transverse ribs 62a, so that the movement of the breast B in the transverse direction is impeded and the slip thereof does not occur.

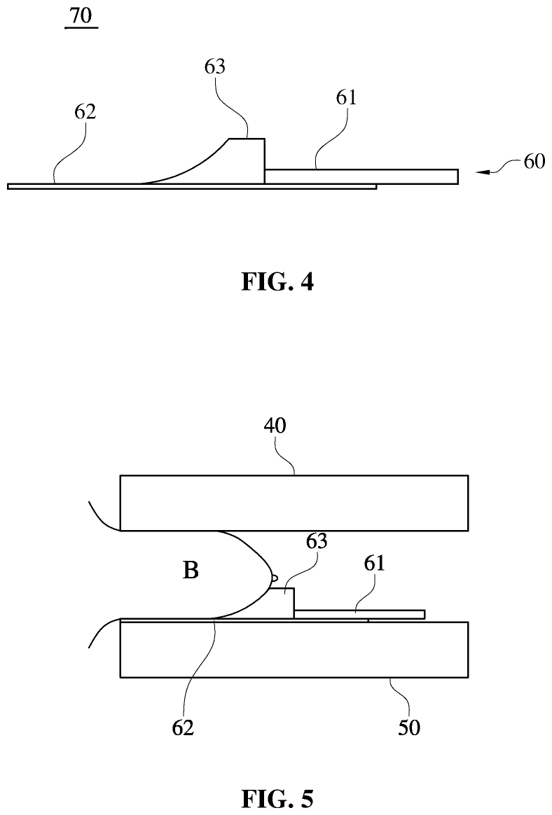

FIG. 4 shows another example of the breast slip prevention pad for the ultrasonic breast scanner according to the present invention.

The breast slip prevention pad 70 of the present embodiment differs from the breast slip prevention pad 60 shown in FIG. 3 in that the breast slip prevention pad 70 further includes a gel pad 63 disposed on one surface of the mesh sheet 62 at a position adjacent to the support plate 61. As shown in FIG. 5, when the breast B is mounted on the mesh sheet 62, the gel pad 63 fills the space between the end portion of the breast B and the scanning unit 50 so that the ultrasonic waves can be smoothly transmitted to the distal end portion of the breast B of the patient. This makes it possible to obtain a clearer image of the breast B. Particularly, the gel pad 63 is configured so as to become thinner as it extends away from the support plate 61 along the transverse ribs 62b, so that an empty space is not formed at the end of the breast B, thereby allowing the ultrasonic waves to be transferred to the distal end portion of the breast.

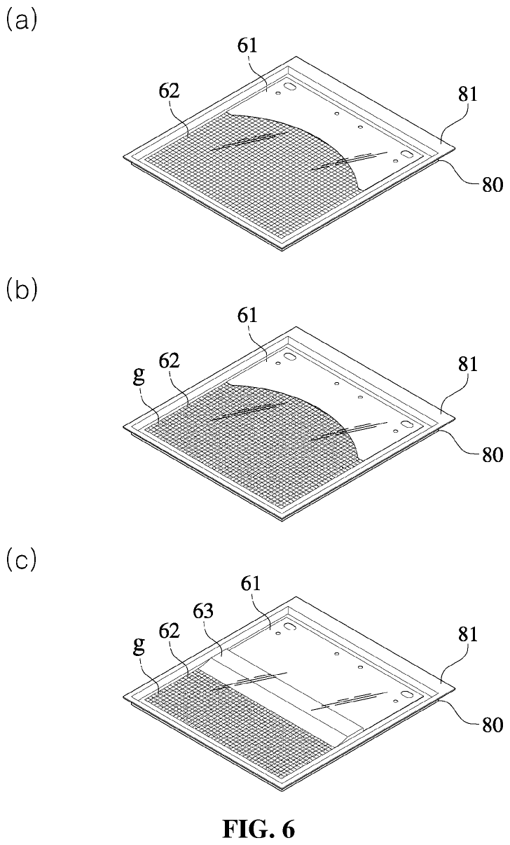

FIG. 6 illustrates explanatory views showing various examples in which the slip prevention pad according to the present invention is packaged in a disposable manner. As shown in FIG. 6A, the breast slip prevention pad 60 may be put into a packaging container 80 and may be sealed with a film 81. Thus, the breast slip prevention pad 60 can be sold in a packaged state against contamination. Furthermore, as shown in FIG. 6B, the breast slip prevention pad 60 may be put into a packaging container 80, a liquid-state gel g may be poured into the container 80, and the breast slip prevention pad 60 may be sealed with a film 81. Thus, the breast slip prevention pad 60 can be sold in a packaged state against contamination. Moreover, as shown in FIG. 6C, the breast slip prevention pad 70 including the gel pad 63 may be put into a packaging container 80, a liquid-state gel g may be poured into the container 80, and the breast slip prevention pad 70 may be sealed with a film 81. Thus, the breast slip prevention pad 70 can be sold in a packaged state against contamination. If a product is supplied in a packaged state as described above, the user can directly use the breast slip prevention pad without having to apply a gel after mounting the breast slip prevention pad on the ultrasonic wave scanner.

It should be understood that the embodiments of the present invention described above are not intended to limit the present invention but are exemplary. The mesh sheet according to the present invention may be modified into various forms. The present invention may be modified and embodied in various forms within the scope of the claims and equivalents thereof.

What is claimed is:

1. A breast slip prevention pad for an ultrasonic breast scanner, comprising:
a mesh sheet formed of an ultrasonic-wave-permeable flexible synthetic resin; and
a support plate fixed to one surface of the mesh sheet to support the mesh sheet in a tightly stretched state so that a part of the mesh sheet is exposed,
wherein the mesh sheet includes a plurality of transverse ribs and a plurality of longitudinal ribs arranged to extend in a direction perpendicular to the transverse ribs, and the height of the longitudinal ribs is set to be larger than the height of the transverse ribs.

2. The pad of claim 1, wherein the transverse ribs of the mesh sheet are arranged to extend toward the support plate.

3. The pad of claim 1, wherein the support plate is provided with a plurality of through-holes.

4. The pad of claim 1, further comprising:
a gel coated on the mesh sheet.

5. The pad of claim 1, further comprising:
a gel pad disposed adjacent to the support plate of the mesh sheet, and
wherein the gel pad is configured to become thinner as the gel pad extends away from the support plate along the transverse ribs.
